(19) United States
(12) Patent Application Publication
(10) Pub. No.: US 2020/0355680 A1
(43) Pub. Date: Nov. 12, 2020

(54) METHOD FOR IDENTIFYING EXOSOME SURFACE MOLECULE

(71) Applicant: HORIBA, LTD., Kyoto-shi, Kyoto (JP)

(72) Inventors: Daisuke IRIKURA, Kyoto (JP); Hideki NAKAYAMA, Kyoto (JP)

(21) Appl. No.: 16/640,894

(22) PCT Filed: Aug. 28, 2018
(86) PCT No.: PCT/JP2018/031798

§ 371 Date: Feb. 21, 2020

(30) Foreign Application Priority Data
Aug. 29, 2017 (JP) 2017-164879
Jul. 13, 2018 (JP) 2018-133709

(51) Int. Cl. G01N 33/543

(52) U.S. Cl. CPC G01N 33/54393

(57) ABSTRACT

The present invention provides a method for identifying an exosome surface molecule, including blocking and washing a carrier having a binding molecule to the exosome surface molecule immobilized thereon with a casein solution or a decomposed casein solution, and mixing a casein solution or a decomposed casein solution and a test sample containing an exosome before contact of the carrier and the test sample.

### TECHNICAL FIELD

The present invention relates to a method for ensuring specific binding of an exosome surface molecule to a binding molecule immobilized on a carrier, and identifying the exosome surface molecule by suppressing non-specific binding of the exosome to the carrier.

### BACKGROUND ART

Currently, the diagnosis of malignant tumors and the like is made by preliminary judgment based on image information such as visual observation, X-ray, CT (Computed Tomography), ultrasound and the like, followed by final judgment based on microscopic observation of the tissue structure using pathological tissue specimens. However, diagnosis based on such information is performed on the basis of a physician's judgment criteria, so that not a little amount of misdiagnosis may occur, which in some cases may lead to a fatal medical accident. To reduce the possibility of misdiagnosis, therefore, information on the abnormality of gene in the suspected tissue and the presence or absence of tumor markers is added, and comprehensive judgment has been made.

Tumor markers have been actively studied in recent years, and refer to tumor-related antigens, enzymes, specific proteins, metabolites, tumor genes, tumor gene products, tumor suppressor genes, and the like. For example, carcinoembryonic antigen (CEA), glycoproteins CA19-9 and CA125, prostate-specific antigen (PSA), calcitonin which is a peptide hormone produced in thyroid gland and the like have been utilized as tumor markers in some cancers for cancer diagnosis. Many tumor markers to be the detection target are body fluid (blood, lymph fluid, urine, etc.) markers, and they can be detected by known means. For example, an immunological detection method includes detection of a tumor marker by utilizing an antigen-antibody reaction. It is a detection method generally rapid, convenient and economical, as well as superior in detection accuracy. In recent years, a surface plasmon resonance (SPR) device capable of measuring a reaction and a binding amount between biomolecules of an antibody and an antigen and performing kinetic analysis by applying a surface plasmon resonance phenomenon and capturing changes in resonance angle in real time has been used in various researches and tests, and has also been applied to tumor marker tests. These methods have a great advantage in that test samples can be processed in a large amount at a low cost by immobilizing an antibody on a carrier.

Incidentally, exosome is becoming a new research trend in the field of tumor research in recent years. Exosome is an extracellular vesicle with a diameter of about 50-150 nm which is secreted from various cells and covered by a phospholipid bilayer membrane. Exosome retains, on the exosome surface and in the exosome, the same molecules (protein, RNA, lipid etc.) as those of the cell that secretes the exosome. Therefore, if a molecule retained by an exosome can be detected as a tumor marker, the exosome can be established as a new method for diagnosing the tumor, and thus is attracting attention. Generally, however, when detecting a molecule retained by the exosome, the membrane structure of the exosome is destroyed and the extracted molecule is directly detected (non-patent documents 1, 2), thus problematically requiring time and labor.

### DOCUMENT LIST

### Non-Patent Documents

non-patent document 1: Jenjaroenpun P et al., PeerJ. Nov. 5; 1:e201, 2013
non-patent document 2: El-Andaloussi S et al., Nat Protoc, 7(12), 2112-26, 2012

### SUMMARY OF THE INVENTION

### Problems to be Solved by the Invention

The present invention aims to provide a method for identifying an exosome surface molecule without destroying the membrane structure of the exosome.

### Means of Solving the Problems

To identify an exosome surface molecule without destroying the membrane structure of the exosome, the present inventors spotted an antibody (anti-c-Kit antibody or negative antibody) on a biochip and immobilized same, then blocked the chip surface with BSA, contacted an exosome previously clarified to retain c-kit as a surface molecule with the chip, and confirmed the reflectance of the both antibodies by using an SPR apparatus. As a result, the reflectance of the both antibodies scarcely changed compared to that before contact. Furthermore, the reflectance of the chip surface part blocked with BSA and other than the part where the antibody was immobilized changed significantly. The present inventors assumed that these results were caused by the facts that exosomes having a phospholipid on the surface were non-specifically bound to BSA since BSA is a lipid binding protein, and that the exosomes could not bind to the antibody because the contacted exosomes were mostly bound non-specifically to the chip surface. Thus, the present inventors have conducted intensive studies in pursuit of a method for ensuring specific binding of an exosome surface molecule to an antibody while suppressing non-specific binding of exosome to the carrier surface. The present inventors spotted the above-mentioned antibody on a biochip and immobilized same, then blocked the chip with a casein solution or a decomposed casein solution instead of BSA, and used a casein solution or a decomposed casein solution instead of BSA as washing to be used for a washing operation. As a result, an increase in the reflectance of the chip surface part blocked with the casein solution or the decomposed casein solution, which is other than the part where the antibody was immobilized, could not be confirmed. Furthermore, an increase in the reflectance of the anti-c-Kit antibody could be confirmed, whereas an increase in the reflectance of the negative antibody could not be confirmed. From these facts, it was found that, by using casein, specific binding of the exosome to an antibody can be ensured while suppressing non-specific binding of exosome to the carrier surface, which resulted in the completion of the present invention.

That is, the present invention provides

[1] a method for identifying an exosome surface molecule, comprising blocking and washing a carrier comprising a binding molecule to the exosome surface molecule immobilized thereon with a casein solution or a decomposed casein solution, and mixing a casein solution or a decomposed casein solution and a test sample containing an exosome before contact of the carrier and the test sample;
[2] a method for identifying an exosome surface molecule, comprising the following steps:
(1) a step of blocking a carrier surface comprising a binding molecule to an exosome surface molecule immobilized thereon with a casein solution or a decomposed casein solution,
(2) a step of washing the carrier with a casein solution or a decomposed casein solution,
(3) a step of contacting a mixture of a test sample containing an exosome and a casein solution or a decomposed casein solution with the carrier,
(4) a step of washing the carrier with a casein solution or a decomposed casein solution, and
(5) a step of detecting binding of the exosome surface molecule and the binding molecule;
[3] the method of [1] or [2], wherein the binding between the exosome surface molecule and the binding molecule is detected by an immunological method or a surface plasmon resonance method;
[4] the method of any one of [1] to [3], wherein the binding molecule is an antibody, a cell adhesion factor, lectin or an aptamer;
[5] a mobile phase comprising casein or decomposed casein for identifying an exosome surface molecule by a surface plasmon resonance method;
[6] an apparatus for identifying an exosome surface molecule for practicing the method of any one of [1] to [4].

### Effect of the Invention

After a binding protein to an exosome surface molecule is immobilized on a carrier, the carrier is blocked with a casein solution or a decomposed casein solution, a casein solution or a decomposed casein solution is used as a buffer to be used for a washing operation, and the casein solution or the decomposed casein solution and a test sample containing exosome are mixed before contact of the carrier and the test sample, whereby specific binding of the exosome to the binding molecule can be ensured while suppressing non-specific binding of exosome to the carrier surface, as a result of which the exosome surface molecule can be identified.

### BRIEF DESCRIPTION OF THE DRAWINGS

FIG. 1 shows the constituent of a microarray SPRi apparatus (Horiba, Ltd.: OpenPlex).

FIG. 2 shows a Flow-cell equipped with a microarray SPRi apparatus (Horiba, Ltd.: OpenPlex).

FIG. 3 shows a biochip (Horiba, Ltd.: CS-HD) exclusive for the microarray SPRi apparatus (Horiba, Ltd.: OpenPlex). The shaded area indicates the portion where the antibody or lectin is immobilized. The hexagonal frame shows where the Gasket in FIG. 2 comes into contact.

FIG. 4 shows changes in the reflectance due to the binding of an anti-c-Kit antibody immobilized on a biochip and c-kit on an exosome surface (conventional method). A: change in reflectance of anti-c-Kit antibody. The change in reflectance shows a difference between the reflectance of anti-c-Kit antibody and the reflectance of goat IgG. B: SPR image shows an image after 600 sec from exosome feeding.

FIG. 5 shows changes in the reflectance due to the binding of an anti-c-Kit antibody immobilized on a biochip and c-kit on an exosome surface (novel immobilization method). A: change in reflectance of anti-c-Kit antibody. The change in reflectance shows a difference between the reflectance of anti-c-Kit antibody and the reflectance of goat IgG. B: SPR image shows an image after 600 sec from exosome feeding.

FIG. 6 shows detection of specific binding between each lectin or each antibody immobilized on a biochip and exosome surface sugar chain or surface antigen. Each photograph shows SPR image in each lectin (ConA; Concanavalin A, SBA; Soybean Agglutinin, MAM; Maackia amurensis, LF; Lectin, Fucose specific from Aspergillus oryzae, SSA; Lectin, sialic acid specific from Sambucus sieboldiana, AAL; Aleuria aurantia Lectin, UEA-I; Ulex Europaeus Agglutinin I, Lotus; Lotus Tetragonolobus Lectin), and each antibody (CD9, CD63, CD81, Mouse IgG's). SPR images show images about 1500 sec after diluted exosome feeding.

### DESCRIPTION OF EMBODIMENTS

The present invention provides a method for identifying an exosome surface molecule, comprising blocking and washing a carrier having a binding molecule to the exosome surface molecule immobilized thereon with a casein solution or a decomposed casein solution, and mixing a casein solution or a decomposed casein solution and a test sample containing an exosome before contact of the carrier and the test sample (hereinafter sometimes to be indicated as the identification method of the present invention).

In the identification method of the present invention, exosome is an extracellular vesicle wrapped in a phospholipid bilayer membrane and secreted from a cell. The cell is not particularly limited and may be an animal cell, a plant cell, a microorganism cell or the like. The animal cell includes mammalian cells and examples of the mammalian cell include, but are not limited to, hepatocyte, splenocyte, nerve cell, glial cell, pancreatic β cell, bone marrow cell, mesangial cell, Langerhans cell, epidermal cell, epithelial cell, goblet cell, endothelial cell, smooth muscle cell, fibroblast, fiber cell, muscle cell, adipocyte, immunocyte (e.g., macrophage, T cell, B cell, natural killer cell, mast cell, neutrophil, basophil, eosinophil, monocyte), megakaryocyte, synovial cell, chondrocyte, osteocyte, osteoblast, osteoclast, mammary cell, or stroma cell, and progenitor cell, stem cell, cancer cell or cultured cell thereof and the like.

In the particular method of the present invention, examples of a surface molecule of exosome (hereinafter sometimes to be simply referred to as surface molecule) include protein, sugar chain, lipid, and the like.

Examples of the protein include membrane protein (integral membrane protein, peripheral membrane protein). Among the membrane proteins, integral membrane protein is preferred, and transmembrane protein is more preferred. As the transmembrane protein, tetraspanin, cell adhesion factor, immunoglobulin superfamily and the like can be mentioned. Examples of the tetraspanin include CD9, CD63, CD81 and the like. Examples of the cell adhesion factor include integrin. Integrin is not particularly limited as long as it is a heterodimer composed of two subunits of α chain and β chain. Examples thereof include integrins α1β1, α2β1, α3β1, α6β1, α7β1, α6β4, α10β1, α11β1, αLβ2, αMβ2, αXβ2, αDβ2, α5β1, αVβ1. αVβ3, αVβ5, αVβ6, αVβ8, αIIbβ3, α4β1, α4β7, α9β1, αDβ2, αLβ2, αMβ2, αXβ2, αEβ7 and the like. Examples of the immunoglobulin superfamily include CD19, EWI-2 and the like.

Examples of the sugar chain include N-glycoside bond type sugar chain, O-glycoside bond type sugar chain and the like.

Examples of the lipid include phospholipid, sphingomyelin, cholesterol, ceramide, lipid raft, glycolipid and the like. Examples of the glycolipid include sphingoglycolipid and the like.

In the identification method of the present invention, the above-mentioned binding molecule to a surface molecule (hereinafter sometimes to be simply referred to as binding molecule) is not particularly limited as long as it can specifically recognize and bind to the surface molecule. For example, protein, nucleic acid can be mentioned.

Examples of the protein include antibody, cell adhesion factor (e.g., integrin), lectin and the like.

Examples of the nucleic acid include aptamer and the like.

The antibody in the particular method of the present invention encompasses both polyclonal antibody and monoclonal antibody. The antibody may encompass antibodies derived from any mammals and may belong to any of the immunoglobulin classes of IgG, IgA, IgM, IgD and IgE, preferably IgG. As the antibody, a commercially available antibody, an antibody stored in a research institute or the like that binds to the target surface molecule may also be used. Alternatively, those of ordinary skill in the art can produce an antibody according to a conventionally-known method.

In addition, the antibody includes naturally-occurring antibodies such as the aforementioned polyclonal antibody, monoclonal antibody (mAb) and the like, a chimeric antibody that can be produced using a gene recombination technique, a humanized antibody, a single-stranded antibody, and fragments of these antibodies. A fragment of an antibody means a partial region of the aforementioned antibody and specifically encompasses Fab, Fab′, F(ab′)2, scAb, scFv, scFv-Fc and the like.

In the identification method of the present invention, the cell adhesion factor may be similar to those described as the exosome surface molecule.

In the identification method of the present invention, lectin is not particularly limited as long as it is a sugar-binding protein or glycoprotein having a property of aggregating cells or composite carbohydrates other than antibody.

In the suppression method of the present invention, examples of the lectin that binds to surface molecule include SBA (Soybean Agglutinin), LCA (Lens culinaris Agglutinin), AAL (Aleuria aurantia Lectin), UEA (Ulex europaeus Agglutinin), PNA (Peanut Agglutinin), WGA (Wheat Germ Agglutinin), Con A (Concanavalin A) and the like.

In the identification method of the present invention, the aptamer refers to a nucleic acid molecule having a binding activity to an exosome surface molecule. The aptamer may be RNA, DNA, modified nucleic acid or a mixture thereof. The aptamer may also be in a linear or cyclic form.

When the aptamer is RNA, a sugar residue (e.g., ribose) of each nucleotide may be modified to enhance stability, drug delivery efficiency, and the like. Examples of the site to be modified in the sugar residue include those in which the hydroxyl group at the 2′-position, 3′-position and/or 4′-position of the sugar residue is replaced with other atom. Examples of the kind of modification include fluorination, alkoxylation, O-allylation, S-alkylation, S-allylation, and amination.

The sugar residue may be a BNA: Bridged nucleic acid (LNA: Linked nucleic acid) having a crosslinked structure at the 2′-position and the 4′-position.

In the identification method of the present invention, the binding molecule is immobilized on a carrier. Immobilization of the binding molecule can be performed by adjusting the above-mentioned binding molecule to a suitable concentration with a buffer, and then spotting the mixture on a carrier and allowing the mixture to stand. The concentration of the binding molecule during immobilization can be appropriately determined and may be, for example, 1 mg/ml. The standing time may be appropriately determined and may be, for example, 8 to 16 hr.

The carrier to be used in the identification method of the present invention is not particularly limited as long as it can be used for immunological method or a surface plasmon resonance method. Examples thereof include synthetic resin such as polystyrene, polyacrylamide, silicon and the like, glass, metal thin film, nitrocellulose membrane and the like.

The identification method of the present invention is characterized in that a carrier on which a binding molecule is immobilized is blocked and washed with a casein solution or a decomposed casein solution. Casein is a phosphorylated protein containing a large amount of highly phosphorylated serine. Since the lipid constituting an exosome is also a phospholipid, Coulomb repulsion occurs between casein and exosome in a solution or on a carrier. Therefore, non-specific binding of exosome to a carrier surface part free of an immobilized binding protein can also be suppressed by blocking the carrier with a casein solution or a decomposed casein solution, and specific binding of an exosome surface molecule to a binding molecule immobilized on a carrier can be simultaneously secured. Blocking with casein can be performed by adjusting casein or decomposed casein with a solvent to a final concentration of 0.1-2%, preferably 1%, filling the surface of the carrier with the solution, and standing the same. As the decomposed casein in the present invention, casein decomposed by acid, casein decomposed by alkali, or casein decomposed by hydrolysis can be mentioned. The solvent is not particularly limited as long as it does not influence the binding between the surface molecule of exosome and the binding molecule. Examples of such solvent include, but are not limited to, distilled water, PBS and the like. The time and temperature for allowing a casein solution or a decomposed casein solution to stand on the surface of the carrier can be appropriately determined by those skilled in the art. For example, the solution can be allowed to stand at room temperature for 10 min to 2 hr. The carrier is washed with a casein solution or a decomposed casein solution. Washing is performed when the carrier is subjected from any step to the next step and, for example, performed when the carrier is blocked with a casein solution or a decomposed casein solution or when the carrier is contacted with a test sample. Washing can be performed by adjusting casein or decomposed casein with a solvent to a final concentration of 0.005-2%, preferably 0.1%, and filling the carrier surface with the obtained solution and allowing same to stand or flow. The solvent may be the same as that mentioned above. The time, temperature, and number of times the casein solution or decomposed casein solution is stood or flown on the carrier surface can be appropriately determined by those skilled in the art. For example, the solution can be stood or flown 1 to 3 times for 10 min to 2 hr at room temperature.

The identification method of the present invention is characterized in that a casein solution or a decomposed casein solution and a test sample are mixed before contact of a carrier and a test sample containing an exosome. Any test sample can be used without particularly limitation as long as it is a sample containing an exosome. The test sample is prepared by subjecting a body fluid (blood, saliva, lacrimal fluid, urine, sweat and the like) of an animal (preferably, mammal) to a centrifugation treatment, density gradient centrifugation, a filter treatment, size-exclusion chromatography, an ultracentrifugation treatment and the like. Using these methods, a test sample having a high exosome concentration can be prepared. The prepared test sample is mixed with a casein solution or a decomposed casein solution (hereinafter mixture). The casein solution or the decomposed casein solution may be the same as the casein solution or decomposed casein solution used for the above-mentioned washing. When the mixture is contacted with a carrier, the time, temperature, and number of times of contact with a surface of the carrier can be appropriately determined by those skilled in the art. For example, the mixture can be contacted 1 to 3 times for 10 min to 2 hr at room temperature.

More particularly, the identification method of the present invention includes the following steps:

(1) a step of blocking a carrier surface having a binding molecule to an exosome surface molecule immobilized thereon with a casein solution or a decomposed casein solution,
(2) a step of washing the carrier with a casein solution or a decomposed casein solution,
(3) a step of contacting a mixture of a test sample containing an exosome and a casein solution or a decomposed casein solution with the carrier,
(4) a step of washing the carrier with a casein solution or a decomposed casein solution, and
(5) a step of detecting binding of the exosome surface molecule and the binding molecule.

In the above-mentioned steps (1)-(5), an exosome, a surface molecule, a binding molecule, a casein solution or a decomposed casein solution, a carrier, a test sample, a blocking method, a washing method and the like may be the same as those described in the identification method of the present invention.

In the particular method of the present invention, the method for detecting the binding of the surface molecule and the binding molecule is not particularly limited. For example, an immunological method and a surface plasmon resonance method can be mentioned.

In the particular method of the present invention, the immunological method is not particularly limited as long as it is an immunological method for detecting a complex composed of a surface molecule and a binding molecule in a test sample by a chemical or physical means, and any measurement method may also be used. In addition, the amount of the surface molecule can also be calculated as necessary from a standard curve drawn using a standard solution containing a known amount of the surface molecule. As the immunological method, any method may be used as long as an antigen-antibody reaction is carried out on the surface of a solid phase, irrespective of a batch system or a flow system, such as ELISA and the like.

As a labeling agent used for a measurement method using a labeling substance, radioisotope, enzyme, fluorescent substance, luminescence substance and the like are used. As the radioisotope [¹²⁵I], [¹³¹I], [³H], [¹⁴C] and the like are used. As the above-mentioned enzyme, one which is stable and having high specific activity is preferable and, for example, β-galactosidase, β-glucosidase, alkaline phosphatase, peroxidase, malic acid dehydrogenase and the like are used. As the fluorescent substance, fluorescamine, fluorescein isothiocyanate and the like are used. As the luminescence substance, luminol, luminol derivative, luciferin, lucigenin and the like are used. In addition, a biotin-avidin system can also be used for binding an antibody and a label.

In a sandwich method, a test sample is reacted with a binding molecule immobilized on a carrier (primary reaction), a labeled secondary antibody to the surface molecule is reacted (secondary reaction), and the amount (activity) of the label on the carrier is measured, whereby the surface molecule in the test sample can be particular. The primary reaction and the secondary reaction may be performed in a reverse order or performed simultaneously or at different times.

Alternatively, using an immunity sensor by a surface plasmon resonance (SPR) method, a binding molecule is immobilized on the surface of a commercially available sensor chip according to a conventional method, it is contacted with a test sample, a light with a particular wavelength is irradiated to the sensor chip from a particular angle, and the presence or absence of binding of the surface molecule to the immobilized binding molecule can be determined with the change in the resonance angle as an index.

Furthermore, in the identification method of the present invention, two or more different binding molecules are immobilized on a carrier to form different configurations, whereby whether respective binding molecules interact with plural surface molecules present in the exosome can be simultaneously verified. For example, it is also possible to contact a test sample with a carrier having an antibody immobilized on at least one spot and lectin immobilized on at least one other spot, and detect interaction of the surface antigen of the exosome in the test sample and the aforementioned antibody, as well as interaction of a sugar chain and the aforementioned lectin. Therefore, the present invention also provides a method for identifying two or more different surface molecules of an exosome, including blocking and washing, with a casein solution or a decomposed casein solution, a carrier on which two or more different binding molecules to two or more different surface molecules of exosome are immobilized such that they are arranged at positions different from each other, and mixing a casein solution or a decomposed casein solution and a test sample containing an exosome before contact of the carrier and the test sample. Also, the present invention provides a method for identifying an exosome surface molecule including the following steps:

(1) a step of blocking, with a casein solution or a decomposed casein solution, a carrier surface on which two or more different binding molecules to two or more different surface molecules of an exosome are immobilized such that they are arranged at positions different from each other,
(2) a step of washing the carrier with a casein solution or a decomposed casein solution,
(3) a step of contacting a mixture of a test sample containing an exosome and a casein solution or a decomposed casein solution with the carrier,
(4) a step of washing the carrier with a casein solution or a decomposed casein solution, and
(5) a step of detecting binding of the two or more different surface molecules of the exosome and the two or more different binding molecules.

By the present method, for example, two or more different surface molecules of an exosome of a test sample can be detected simultaneously, and therefore, the test sample can be diagnosed rapidly. Specifically, in cancer diagnosis, since sugar chain and surface antigen on an exosome surface can be detected simultaneously, the diagnosis thereof can be performed rapidly.

The present invention also provides a mobile phase containing casein or decomposed casein for identifying an exosome surface molecule by a surface plasmon resonance method (hereinafter sometimes to be indicated as the mobile phase of the present invention). In the present invention, the mobile phase refers to a solution containing casein or decomposed casein used for washing a carrier or mixing with a test sample in the identification method of the present invention. Casein or decomposed casein may be the same as that described in the identification method of the present invention. Examples of the solvent for dissolving casein or decomposed casein include, but are not limited to, distilled water, PBS and the like.

Casein or decomposed casein provided as the above-mentioned mobile phase may be a dry powder or a solution obtained by dissolving in distilled water, PBS to a suitable concentration. In the case of a solution, it can be preserved at about −20° C.

The present invention also provides an identification apparatus of an exosome surface molecule for performing the identification method of the present invention (hereinafter sometimes to be indicated as the apparatus of the present invention). The apparatus of the present invention includes a microarray SPRi apparatus and biochip. The biochip is composed of a prism and a metal to be formed into a film on one side of the prism. The shape of the prism includes a trapezoid, a triangle, a circle (semicircle) and the like. The refractive index of the prism is generally 1.5-1.8. As the metal to be formed into a film on one side of the prism, gold, silver, copper, aluminum and the like can be mentioned. The surface of the biochip preferably has a carboxy group activated with succinimide and immobilized on the surface thereof. The microarray SPRi apparatus is provided with a sensor that detects a reflected light associated with the SPR phenomenon induced by the binding of exosome to the biochip surface, and a device that calculates and outputs the amount of change in the reflected light as reflectance (%). The above-mentioned microarray SPRi apparatus is also provided with a device that converts change in the calculated reflectance into a color tone image and outputs same. The apparatus of the present invention can also confirm changes in the color tone of a biochip surface where the binding molecule is not immobilized, and thus can confirm the presence or absence of non-specific binding.

### EXAMPLES

While the present invention is explained more specifically in the following by referring to Examples, the invention is not limited to them.

### Construction of Exosome Detection Biosensor by Surface Plasmon Resonance (SPR)

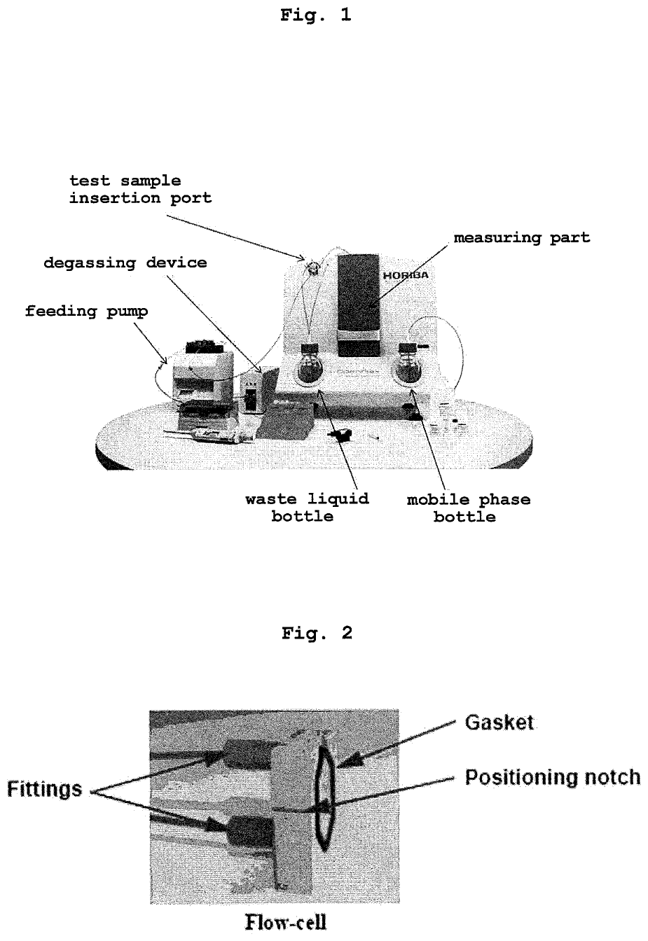

An exosome detection biosensor by surface plasmon resonance (SPR) was constructed using a microarray SPRi apparatus (Horiba, Ltd.: OpenPlex) (FIG. 1) and a biochip exclusive for the apparatus (Horiba, Ltd.: CS-HD; biochip on which carboxy group activated by succinimide is immobilized). The constructed sensor can measure every 3 sec the amount of change in reflected light due to the SPR phenomenon induced by the binding of exosome to the chip surface as reflectance (%). At the same time, the change of reflectance of SPR can be observed as a spot image. The chip has a surface area of 12 mm×23 mm and thus can characteristically arrange many spots in parallel by adjusting the spot diameter (spot amount) of the ligand solution for immobilization. The microarray SPRi apparatus used in this Example is provided with a measuring part including a biosensor for detecting exosome, a mobile phase bottle for storing mobile phase for identifying exosome surface molecule, a waste liquid bottle for storing waste liquid containing a test sample after completion of detection, a pump for feeding a test sample or a mobile phase, a degassing device for degassing a mobile phase, and a test sample insertion port.

### Comparative Example Detection of Exosome with Biochip Bound with Antibody (Conventional Method: Blocking by BSA)

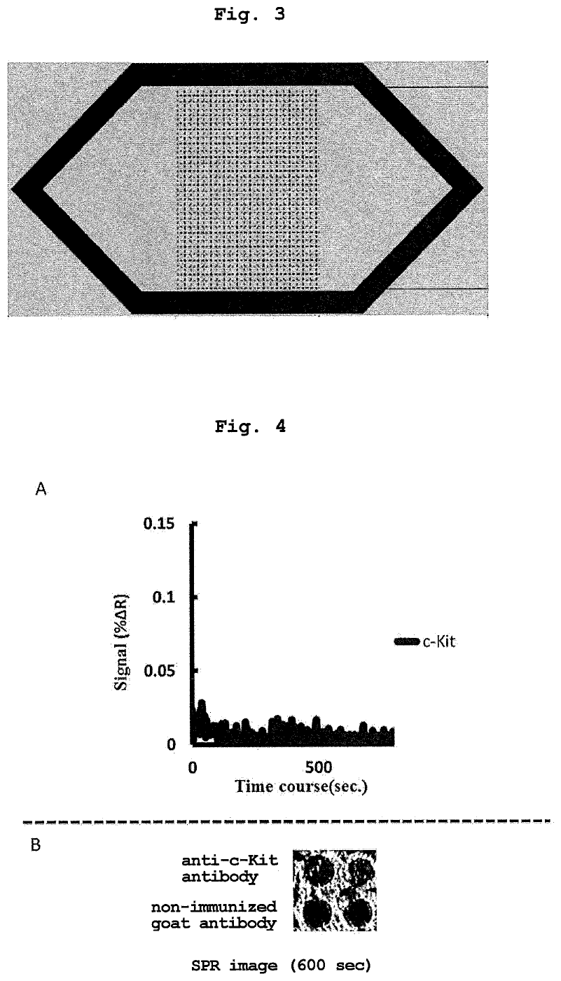

As the exosome, exosome released by a mouse bone marrow-derived mast cell was used. The exosome is known to have c-Kit on a surface thereof. For exosome detection, an antibody against surface antigen c-Kit (anti-c-Kit antibody; R&D systems Inc., AF1356) and a non-immunized goat antibody (goat antibody; Abcam Inc., ab37373) as a negative antibody were used. As the antibody, a non-immunized goat antibody (Abcam Inc., ab37373) was used. The antibody was spotted by 10 nL on a chip surface with a spotter and immobilized by standing for 16 hr. The chip surface was washed with Dulbecco's PBS(-) (hereinafter to be abbreviated as PBS), filled with PBS containing 1% BSA dissolved therein, and stood for 1 hr at room temperature for blocking. The blocked chip was washed 3 times with PBS and mounted on the apparatus. The buffer or sample was contacted with the chip surface via Flow-cell (FIG. 2). Flow-cell is fixed in contact with the chip in a position (FIG. 3) where the entire Gasket is completely covered by the chip. Among the flat planes of Flow-cell, the flat plane surrounded by the frame of Gasket is recessed by 80 μm than the flat plane of the periphery of the Gasket frame. As a result, in the chip in contact with the Flow-cell, a spatial gap of 80 μm in width is generated between the flat plane surrounded by the Gasket frame of the Flow-cell and the chip surface. Therefore, a buffer or the like fed from one polyvinyl chloride tube (inner diameter 380 μm) connected to the Flow-cell via Fitting contacts the surface of the chip by filling the spatial gap of 80 μm in width, and excreted from the other polyvinyl chloride tube. PBS (buffer A) as a running buffer (referring to the above-mentioned mobile phase) was supplied to the device equipped with the chip at a flow rate of 25 μL/min to condition the chip surface. Assuming that the reflectance at the time of stabilization was 0%, exosome was suspended in buffer A, fed for 480 sec, and immediately thereafter, buffer A alone was fed for 480 sec, and the antibody reflectance was measured over time. As a result, specific binding of the exosome to the anti-c-Kit antibody could not be detected from the difference obtained by subtracting non-immunized goat antibody reflectance from anti-c-Kit antibody reflectance (FIG. 4A). As is also clear from the SPR image, the part immobilized with the anti-c-Kit antibody scarcely showed changes in the color tone, and the color tone of the part blocked with BSA and other than the part where antibody was immobilized changed (FIG. 4B), from which it was clear that exosome was bonded to BSA. This indicates that BSA cannot suppress non-specific binding of exosome to the chip, but rather causes non-specific binding. In addition, it is shown that exosome could not bind to the antibody because most of the contacted exosomes were non-specifically bound to the chip surface, due to which the color tone of the part where the anti-c-Kit antibody was immobilized scarcely changed. Furthermore, when the concentration of the immobilized antibody is low, it is assumed that the inside of the spot where the antibody is immobilized is also blocked with BSA, and when detecting exosomes, they bind not only to the antibody but also to BSA, and show false-positive and false-negative. Therefore, to establish a detection system for exosome surface molecules which uses antibody, it was found that suppression of non-specific binding of exosome by a method not using BSA is necessary.

### Example 1 Detection of Exosome by Biochip Bonded with Antibody (Novel Measurement Method: Blocking with Casein)

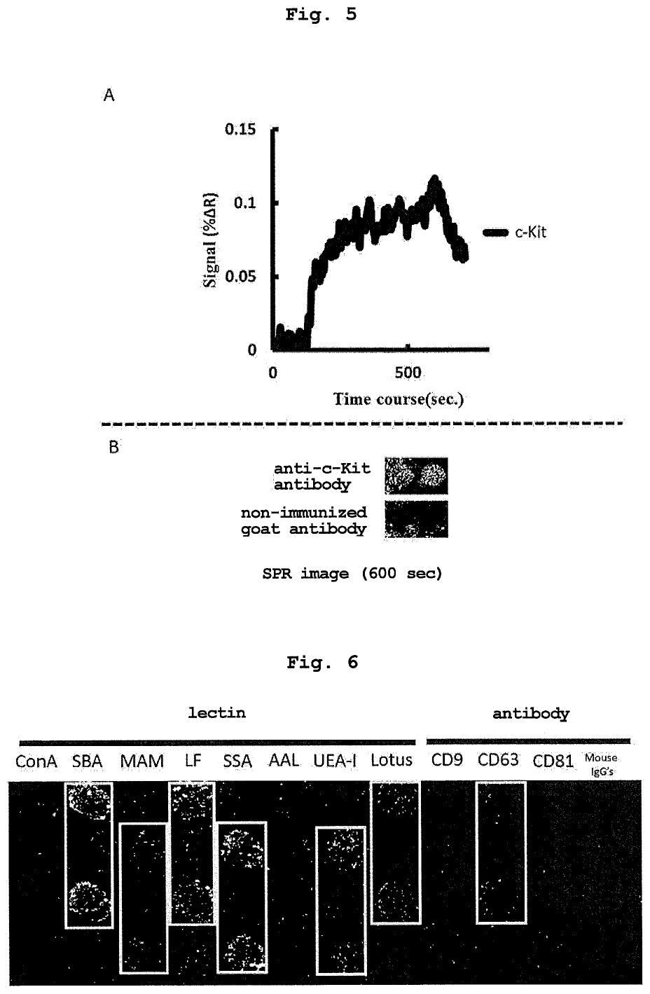

Similar to Comparative Example, exosome released by mouse bone marrow-derived mast cell was used as the exosome. Also, similar to Comparative Example, an antibody against surface antigen c-Kit was used for exosome detection (anti-c-Kit antibody; R&D systems Inc., AF1356) and a non-immunized goat antibody (goat antibody; Abcam Inc., ab37373) was used as a negative antibody. Biochip was produced using the same reagents and method as in Comparative Example except for blocking. Blocking was performed by filling a chip surface with 1% casein dissolved in PBS and standing same for 1 hr at room temperature. The blocked chip was washed 3 times with PBS and mounted on the apparatus. The apparatus mounting chip was fed with PBS (buffer B) containing 0.1% casein as a running buffer (referring to the above-mentioned mobile phase) at a flow rate of 25 μL/min, and the chip surface was conditioned. The reflectance at the time point of stabilization was taken as 0%, exosome suspended in buffer B was fed for 480 sec, and immediately thereafter, buffer B alone was fed for 220 sec, and the antibody reflectance was measured over time. As a result, the reflectance of the anti-c-Kit antibody increased to about 0.1% at maximum and the reflectance of the negative antibody did not increase (FIGS. 5A, 5B). As a result, compared to BSA used in Comparative Example, casein enabled specific binding between the surface molecule of exosome and the antibody against the surface molecule. Also in the SPR image at 600 sec after the start of feeding shown in FIG. 5B, the above-mentioned specific binding could be easily observed. That is, in the part where c-Kit antibody was immobilized, the color tone changed along with an increase in the reflectance, and the color tone did not change in the part where a non-immunized goat antibody was immobilized. In addition, the color tone scarcely changed in the chip surface part blocked with BSA and other than the part where the antibody was immobilized. From these results, it was clarified that blocking with 1% casein and addition of 0.1% casein to the feeding buffer suppressed non-specific binding of exosome to a chip surface other than the part where an antibody was immobilized, as a result of which physical contact between the surface molecule of the exosome and the antibody against the surface molecule increased and specific interaction could be observed.

### Example 2 Simultaneous Detection of Sugar Chain and Surface Antigen of Human Serum-Derived Exosome by SPR Image Method

Surface antigen that is membrane protein and sugar chain are present on the cell surface in addition to lipids that form cell membrane. Surface antigen is responsible for cell activation as a corresponding ligand or a receptor for outside stimulation. In addition, it is known that, after differentiation or maturation of cell by a ligand or outside stimulation, the sugar chain changes its sequence and becomes a target molecule. For example, microorganism and virus recognize a specific cell surface sugar chain and infect or invade cells. In the process of canceration of normal cells, the expression of cancer cell-specific sugar chain and the expression of specific sugar chain increase, and the surface sugar chain sequence of exosomes released by these cells also changes. Therefore, sugar chain can be expected as a useful biomarker for distinguishing microorganism, cell and exosome. In fact, in clinical settings, surface antigens and sugar chains are used as biomarkers. Surface antigens are mainly analyzed by a flow cytometer. However, sugar chain analysis has a complicated structure and is sensitively affected by many environmental factors, and analysis in a short time by a structural change or a DNA sequence is not available. Therefore, an analysis method for sugar chain is complicated and very difficult. For this reason, simultaneous detection of a surface antigen, which is a membrane protein, and sugar chain analysis has not been performed at present. In this example, therefore, simultaneous detection of sugar chain and surface antigen was performed using, as an analyte, human purified exosome assuming a human sample, and using, as a ligand, lectin that is a protein specifically recognizing a sugar chain sequence or a surface antigen-specific antibody. As a detection method, an SPRi method capable of simultaneously detecting multiple samples was used.

As human serum-derived exosomes used as analytes, purified by using Human Serum (S4200-100) (10 ml) manufactured by Biowest and exosome isolation kit PS (293-77601) manufactured by Fujifilm Wako Pure Chemical Corporation and according to the protocols thereof. As the ligand, 8 kinds of Concanavalin A (ConA; Nacalai Tesque, 09446-94), Soybean Agglutinin (SBA; J-chemical, J117), Maackia amurensis (MAM; J-chemical, J110), Aspergillus oryzae-derived purified fucose-specific lectin (LF; Tokyo Chemical Industry Co., Ltd., L0169), Sambucus sieboldiana-derived purified sialic acid-specific lectin (SSA; J-chemical, J118), Aleuria aurantia Lectin (AAL; J-chemical, J101-R), Ulex europaeus Agglutinin I (UEA-I; J-chemical, J119), Lotus tetragonolobus Lectin (Lotus; J-chemical, J109) were used for exosome sugar chain detection. In addition, 3 kinds of CD9 antibody (CD9; R&D systems Inc., MAB1880), CD63 antibody (CD63; Santa Cruz Biotechnology, sc-365604), CD81 antibody (CD81; Santa Cruz Biotechnology Inc., sc-166029), which are tetraspanin antibodies, were used for exosome surface antigen detection. As a negative control, mouse antibody (Mouse IgG's; Sigma-Aldrich Inc., 18765) was used. 0.1% Gelatin having a suppressive effect on the non-specific binding between each of the aforementioned ligands and exosome was mixed with each of the aforementioned ligands, and the mixture was spotted by 10 nL on a chip surface with a spotter and allowed to bind by standing for 16 hr. The chip surface was washed with PBS, the chip surface was filled with 1% casein, and blocked by being stood for 16 hr at room temperature. The blocked chip was washed 3 times with PBS and mounted on the apparatus. The apparatus was fed with PBS (buffer A) containing 0.1% casein as a running buffer at a flow rate of 25 μL/min, and the reflectance at the time point of equilibration of the chip surface was taken as 0. Next, the purified exosome was diluted with buffer A to 10-fold dilution. The diluted exosome (200 μL) was injected into the apparatus and fed for 240 sec. Since the binding rate between the exosome and lectin was slow and the binding was inhibited by the flow of the liquid, the feeding was temporarily stopped, and the exosome diluted solution was kept on the chip surface for 600 sec, whereby the exosome and lectin were bound and aggregated. Thereafter, buffer A alone was further fed for 240 sec, and a total of 1080 sec was taken as the binding process. Thereafter, as a dissociation process, buffer A alone was fed for 480 sec to wash the surface of the biochip.

As a result, in the SPR image after about 1500 sec in the dissociation process by substituting with buffer A, positive lectins are SBA, MAM, LF, SSA, UEA-I, Lotus, and as for the antibody, CD63 was positive and Mouse IgG's was negative (FIG. 6). The above results simultaneously reveal that α-bound fucose and sialic acid-containing N- or O-type sugar chain and lipid-bound sugar chain are present on purified exosome, and that CD63 is present as tetraspanin, a surface antigen. In addition, since the negative control, Mouse IgG's, was negative, the measurement system was established.

### INDUSTRIAL APPLICABILITY

Using the identification method of the present invention, information used for diagnosis of malignant tumor and the like can be obtained from exosome. This application is based on patent application No. 2017-164879 filed in Japan (filing date: Aug. 29, 2017) and patent application No. 2018-133709 filed in Japan (filing date: Jul. 13, 2018), the contents of which are incorporated in full herein.

1. A method for identifying an exosome surface molecule, comprising blocking and washing a carrier comprising a binding molecule to the exosome surface molecule immobilized thereon with a casein solution or a decomposed casein solution, and mixing a casein solution or a decomposed casein solution and a test sample containing an exosome before contact of the carrier and the test sample.

2. A method for identifying an exosome surface molecule, comprising the following steps:
(1) a step of blocking a carrier surface comprising a binding molecule to an exosome surface molecule immobilized thereon with a casein solution or a decomposed casein solution,
(2) a step of washing the carrier with a casein solution or a decomposed casein solution,
(3) a step of contacting a mixture of a test sample containing an exosome and a casein solution or a decomposed casein solution with the carrier,
(4) a step of washing the carrier with a casein solution or a decomposed casein solution, and
(5) a step of detecting binding of the exosome surface molecule and the binding molecule.

3. The method according to claim 1, wherein the binding between the exosome surface molecule and the binding molecule is detected by an immunological method or a surface plasmon resonance method.

4. The method according to claim 1, wherein the binding molecule is an antibody, a cell adhesion factor, lectin or an aptamer.

5. A mobile phase comprising casein or decomposed casein for identifying an exosome surface molecule by a surface plasmon resonance method.

6. An apparatus for identifying an exosome surface molecule for practicing the method according to claim 1.
